(19) United States
(12) Patent Application Publication
(10) Pub. No.: US 2002/0099219 A1
(43) Pub. Date: Jul. 25, 2002

(54) Piperidine compounds and process for providing the same

(76) Inventors: Jacobus M. Lemmens, Mook (NL); Theodorus H.A. Peters, Arnhem (NL); Pavel Slanina, Lelekovice (CZ)

Correspondence Address: SYNTHON PHARMACEUTICALS, LTD., PATENT DEPT., P.O. BOX 161, CATHARPIN, VA, 20143, US

(21) Appl. No.: 09/853,860

(22) Filed: May 14, 2001

(30) Foreign Application Priority Data
May 12, 2000 (NL) PCT/NL/00/00321

(51) Int. Cl. C07D 211/08; A61K 31/445

(52) U.S. Cl. 546/236; 514/317

(57) ABSTRACT

The tosylate ester of the formula (6) and its salts, are convenient intermediates in the synthesis of paroxetine.

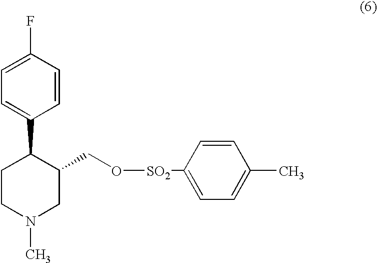

This application claims the benefit of priority under 35 U.S.C. § 119 from PCT/NL/00321, filed May 12, 2000, the entire contents of which are incorporated herein by reference.

### BACKGROUND OF THE INVENTION

The present invention relates to a piperidine compound useable in producing paroxetine, and to a process for making this compound and for using this compound to make paroxetine.

(−)Trans-4-(p-fluorophenyl)-N-methyl-piperidine-3-carbinol of the general formula (1)

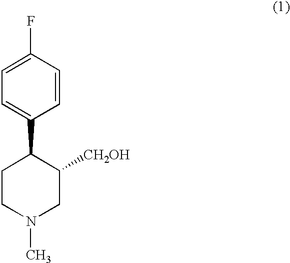

is a starting material in the synthesis of the pharmaceutically active compound paroxetine, represented by the formula (2).

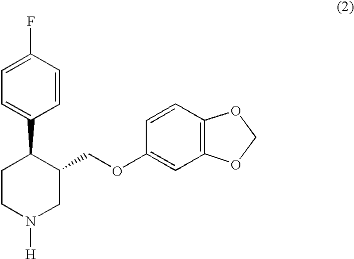

A known and useful synthetic procedure leading to paroxetine, formula (2), is outlined in the following scheme:

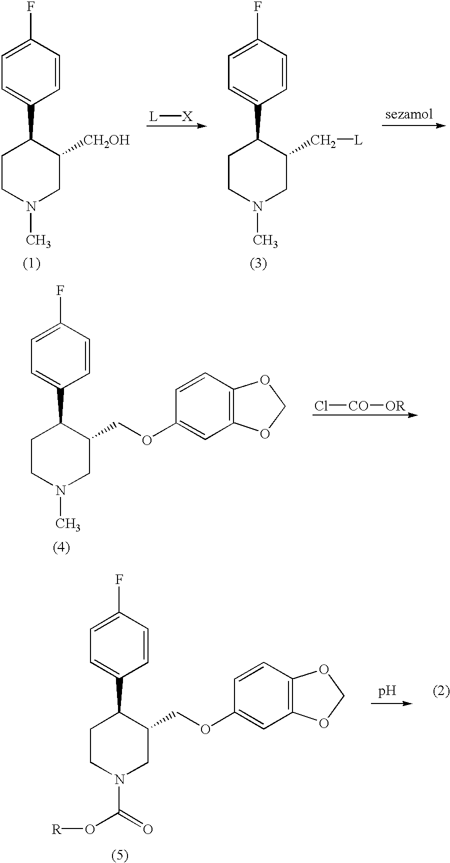

The scheme comprises the following reaction steps:

(a) The —OH group of compound (1) is substituted by a leaving group L;

(b) The reactive substrate (3) so produced reacts with an anion of sesamol (3,4-methylenedioxyphenol) to yield N-methylparoxetine (4);

(c) To remove the methyl group, the compound (4) is reacted with a chloroformate Cl—COOR to yield a carbamate compound (5). Several chloroformates with different R groups have been disclosed in the prior art as suitable; and

(d) The carbamate group in (5) is hydrolysed to paroxetine (2) in a strongly alkaline or acidic medium, dependent on the nature of the R substituents. For R=phenyl, alkaline conditions are preferred.

The leaving group L is so selected that it is reactive in the subsequent synthetic step. Known leaving groups include a chlorine atom or a sulfonic acid ester group such as a mesyloxy or besyloxy group. The compound (3) wherein the L group represents a mesyloxy group or a besyloxy group have been generally favored as these compounds can be produced in good yields and without the use of corrosive and irritating substances such as thionylchloride.

The known intermediates (3), according to the prior art are prepared in situ to be directly used in the reaction with sesamol. In attempts to isolate the product, i.e., after elaboration of reaction mixtures and evaporation of the solvent, the mesyloxy or besyloxy compound results in an oil. This represents a disadvantage. Oily materials, produced in a synthesis, are likely to be contaminated with solvent and reagent residue from the synthesis which may cause side reactions in subsequent steps. Further, in this particular scheme, the starting carbinol (1) is usually contaminated with the corresponding des-fluoro impurity. If so, the process leading to the known intermediates (3) may not provide the intermediate (3) in the desired purity. As a result, the produced paroxetine also contains an undesirably high amount of the correspondingly formed des-fluoroparoxetine. If paroxetine is used as a pharmaceutical substance, the content of the des-fluoro paroxetine therein should be less than 0.1% according to Pharmacopoeia prescriptions (e.g. Paroxetine hydrochloride USP XXIII). Purifying of crude paroxetine contaminated with the des-fluoroparoxetine is ineffective and uneconomical.

It would be advantageous to provide a compound of formula (3) that could be isolated as a solid, preferably crystalline material, for synthetic convenience and/or quality control/quality assurance reasons. Further, it would be advantageous to provide a compound of formula (3) that contains low amounts of the corresponding des-fluoro impurity and/or that is readily purified with respect to the des-fluoro impurity.

### SUMMARY OF THE INVENTION

A first aspect of the present invention relates to the compound (−)trans 4-(p-fluorophenyl)-3-(p-toluenesulfonyloxymethyl)-N-methylpiperidine of the formula (6).

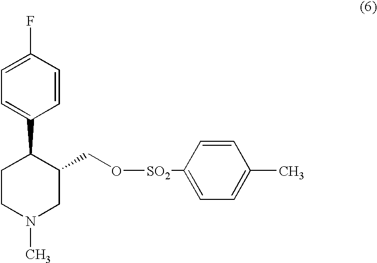

Compound (6) can be isolated in a solid form and accordingly has good handling properties, and is stable during prolonged storage and/or transport. Furthermore this solid form has a low content of the corresponding des-fluoro impurity, and is easily purified. The solid state provides advantages for transport sampling and weighing during industrial chemical production as well as for identification, analytical and purification procedures, which are extremely important in the production of pharmaceuticals.

A second aspect of the present invention relates to a method for making the compound of formula (6) which comprises contacting the compound of formula (1) with a tosyl moiety-providing compound to form a compound of formula (6).

A third aspect of the invention relates to a method of using the compound of formula (6) to make paroxetine or pharmaceutically acceptable salts thereof, which comprises converting said compound of formula (6) to paroxetine or a pharmaceutically acceptable salt thereof.

### DETAILED DESCRIPTION OF THE INVENTION

The compound of formula (6) according to the present invention is not limited in physical form and can be obtained and used dissolved in solution, as a crystalline solid, etc. Crystalline forms include hydrates and solvates, depending on the solvent system, isolation technique, washing and drying conditions, etc. The compound of formula (6) also includes salts thereof such as pharmaceutically acceptable salts. The compound (6) is sufficiently stable towards heat, moisture and light that it can be stored and transported at standard storage/transport conditions. It may be handled (weighed, packed, sampled, charged in the reaction vessel etc.) under common protective precautions, without the need for special equipment.

The compound (6) has been identified and characterised by one or more of the following procedures: elemental analysis, nuclear magnetic resonance spectroscopy (NMR), infrared spectroscopy (IR) and high performance liquid chromatography (HPLC).

The process for making the compound of formula (6) can be carried out using known reagents and known or readily determinable conditions by workers skilled in the art. The reaction involves contacting a compound of formula (1) with a tosyl moiety-providing compound. A “tosyl” moiety is a p-toluenesulfonyl as shown in formula (6) and corresponds to the “L” group of the prior art process described in Scheme 1. A tosyl moiety-providing compound is, as the name indicates, any compound that can provide a tosyl moiety. Typically the tosyl moiety-providing compound is one that contains a tosyl moiety bonded to a leaving group, especially a leaving group that renders the reagent suitable for a substitution reaction with the compound of formula (1). Examples of suitable tosyl moiety-providing compounds include p-toluenesulfonylhalides especially p-toluenesulfonylchloride (also known as tosyl chloride), as well as tosylate anhydride (dimeric tosyl compound). The compound of formula (1) can be made by a variety of techniques; a conventional method being set forth in U.S. Pat. No. 4,902,801.

The reaction is generally carried out in an inert solvent. The solvent is typically an organic solvent such as an alkanol and an acid ester. Preferably the solvent is ethyl acetate or a lower alcohol such as ethanol or isopropyl alcohol. The solvent is preferably anhydrous. The reaction is normally carried out in the presence of a base, especially an organic base. Preferably the base is triethylamine or pyridine, although the reaction is not limited thereto. The base is generally present in the reaction medium and usually it is used in a slight excess. The reaction can be carried out at a variety of temperatures and is not particularly limited in this respect. Generally the reaction is carried out in the range from 0 to 80° C., more typically from 20 to 70° C., and preferably about 60° C.

The compound (6) is generally isolated from the reaction mixture as a precipitate by filtration or centrifugation. The obtained solid may be washed with an appropriate washing liquid and dried. Usually the precipitated compound (6) is in the form of a crystalline material. Interestingly, while both the compound of formula (1) and the reaction product are normally colored, the isolated compound of formula (6) is normally a white or colorless material.

The starting compound (1) usually contains a certain amount of the structurally related des-fluoro impurity which reacts in the same way as (1) and consequently produces the des-fluoro impurity of formula (3). The content of the des-fluoro impurity in (1) can be undesirably high, especially when (1) has been prepared using the most convenient hydride reduction method. In particular, raw (1) so prepared can contain 1-2% of the des-fluoro impurity. When the reactive intermediate (3) produced from (1) is used in situ as in the prior art methods, it has been found that the content of des-fluoro impurity in the subsequent steps of paroxetine production remained basically the same as that in the starting (1) material. Thus, in the conventional process scheme, once the des-fluoro impurity was present, it was difficult to remove. Advantageously, the compound (6) according to the present invention, when isolated from the reaction medium as a solid, contains, without employing any purification technique, considerably less des-fluoro impurity than the starting compound (1). Preferably, the isolated compound (6) contains less than 0.2% of the corresponding des-fluoro impurity.

Moreover, the content of the des-fluoro impurity in the isolated compound (6) material may be further decreased, if desired and/or necessary, by subsequent recrystallization of (6) from a suitable solvent. Suitable solvents with regard to purification and yield preferably comprise lower alkanols such as methanol, ethanol or most preferably isopropanol. In general, crystallization of (6) from ethanol exhibits a purification effect (decrease of des-fluoro impurity) of 16-25% with 85-95% yield, while isopropanol provides 35-40% purification effect in 85-90% yield. Ethyl acetate generally provides good yields in recrystallization but with almost 0% purification effect. Recrystallization can be done one or more times depending on the level of impurities initially present, the purification effect, and the desired purity level. Alternatively, or in addition thereto, the compound of formula (6) may be isolated as, or converted to, an acid addition salt. A good crystallizing salt is the tosylate salt which can be prepared from (6) and p-toluene sulfonic acid in an aqueous medium and crystallised from the same medium in almost 100% yield. The purification effect of such conversion is considerable, although lower than recrystallizing from ethanol (generally about 10-15%).

Crystalline forms of the compound of formula (6) thus preferably contain 0.2% or less, more preferably 0.1% or less, of the corresponding des-fluoro impurity.

The compound (6) is preferably converted to paroxetine by the known general synthetic route as is illustrated by Scheme 1 above. The resulting paroxetine can be used as the free base or as a pharmaceutically acceptable salt; i.e. paroxetine hydrochloride, paroxetine mesylate, etc. Paroxetine and its pharmaceutically acceptable salts can be made by the present invention with a low amount of paroxetine des-fluoro impurity due to the purity of the compound of formula (6).

The following scheme illustrates a detailed process comprising steps according to the present invention:

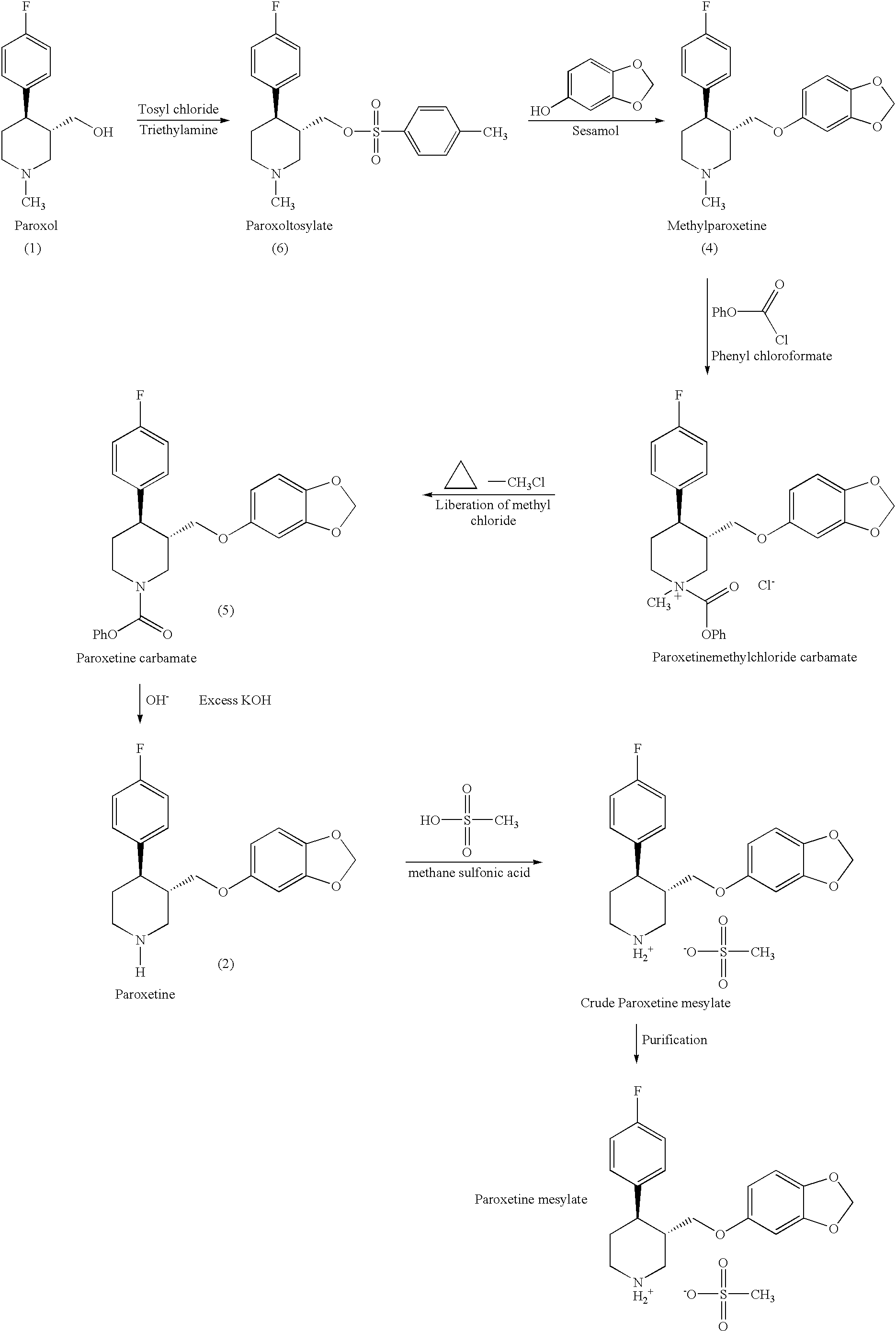

The compound (6) referred to as “paroxoltosylate”of the present invention is preparable by a process starting from a compound of formula (1), referred to as “paroxol” in this scheme.

The method of conversion of (1) to (6) preferably comprises the reaction of (1) in an inert solvent with p-toluenesulfonyl chloride (tosyl chloride) in the presence of an organic base. The resulting hydrogen chloride is trapped by the base. The solvent is preferably inert to tosyl chloride in order to prevent solvolysis, and, should dissolve, at least partly, the reactants and the product to crystallize therefrom.

In the preferred process, the carbinol (1) “paroxol” in ethyl acetate is reacted with tosyl chloride in the presence of 1.0-1.5 molar amount of triethylamine or pyridine at a temperature close to ambient or slightly elevated (from 0 to 80° C., preferably around 60° C.). Care is generally taken that the solvent and reagents are substantially anhydrous. The triethylamine or pyridine hydrochloride resulting from the reaction is insoluble in the reaction medium and is easily removed after completion of the reaction by filtration, preferably at elevated temperature. After cooling the clear filtrate, the product (6) crystallizes spontaneously. If desired, the clear filtrate may be slightly concentrated prior to crystallisation, e.g. on a rotary vacuum evaporator. The precipitated compound (6) is isolated from the reaction mixture by filtration or centrifugation; optionally with washing and drying as mentioned above.

The above method of Scheme 2, optionally in combination with one or more recrystallizations, can provide the compound (6) and/or its acid addition salt with the content of less than 0.2% of the des-fluoro impurity. If desired the starting paroxol can be purified with respect to des-fluoro paroxol impurity by crystallization of the tosylate salt thereof as is more fully described in commonly owned co-pending U.S. patent application Ser. No. ______, (Atty Docket No. POT-012US) filed May 14, 2001, entitled “Tosylate Salts of 4-(p-Fluorophenyl)-Piperidine-3-Carbinols” by Lemmens et al, the entire contents of which are incorporated herein by reference. In any event, this particular quality of (6) allows the production of paroxetine in a pharmaceutically acceptable quality without the need of employing complicated, expensive and time consuming specific purification steps down stream focused to des-fluoro impurity removal.

The amount of the structurally related des-fluoro impurities in products (1) and (6) can advantageously be monitored by HPLC, preferably using a reference substance of the des-fluoro impurity prepared according to methods known per se.

The compound of formula (6), particularly in a solid state and optionally recrystallized, can be further converted into paroxetine of formula (2) for example employing procedures outlined in prior art disclosures, e.g. in U.S. Pat. Nos. 4,007,196 and 4,721,723, which may then be further processed to other pharmaceutically acceptable acid addition salts, such as paroxetine mesylate, as shown in the Scheme 2. Paroxetine or a pharmaceutically acceptable salt thereof can be formulated into pharmaceutical compositions by combining an effective amount of the paroxetine compound with a pharmaceutically acceptable excipient, as is well known in the art. Typically the paroxetine is used in a 10, 20, 30, or 40 mg unit dose.

The following examples illustrate the invention but it should be understood that the present invention is by no means restricted to these specific examples.

### EXAMPLES

### Example 1

### (−)trans-4-(p-fluorophenyl)-3-(p-toluenesulfonyloxymethyl)-1-methyl-piperidine, Compound (6)

5.2 g of (−)trans-4-(p-fluorophenyl)-N-methyl-piperidine3-carbinol, “paroxol” was added under stirring to a mixture of 25 ml of ethyl acetate and 3.7 ml of triethylamine. After dissolution, the mixture was cooled to 0-5C and a solution of 4.7 g of p-toluene sulfonylchloride in 9.5 ml of ethyl acetate was added dropwise at the same temperature within 1 hour.

The reaction mixture was stirred at the same temperature for 1 hour and then for 24 hours at ambient temperature. The mixture was then heated to 60° C. and filtered hot. The filtration cake was washed with 9 ml of ethyl acetate. The combined filtrates were concentrated in vacuo to approx. 25 ml volume, the concentrate was cooled under stirring to 0-5° C. and maintained at the same temperature for 24 hours.

The resulting crystals were filtered off, washed with 10 ml of cold ethyl acetate and dried. Yield 7.3 g.

The compound was identified by NMR and IR spectra, characterised by m.p. and optical rotation, the purity was determined by HPLC.

### Example 2

### Recrystallization of (−)trans-4-(p-fluorophenyl)-3-(p-toluenesulfonyloxymethyl)-1-methyl-piperidine, Compound (6)

7.3 g of the compound from Example 1 was dissolved in 11.5 ml of isopropanol at 60° C. and the resulting solution was slowly cooled to 20° C. When the crystallization started, the mixture was cooled to 0-5° C. and maintained at the same temperature for 24 hours. The solid product was filtered off, washed with 10 ml of cold isopropanol and dried. Yield: 6.6 g.

The invention having been described, it will be readily apparent to those skilled in the art that further changes and modifications in actual implementation of the concepts described herein can easily be made or may be learned by practice of the invention, without departing from the spirit and scope of the invention as defined by the following claims.

1. A (−)trans-4-(p-fluorophenyl)-3-(p-toluenesulfonyloxymethyl)-1-methyl-piperidine of the formula (6), 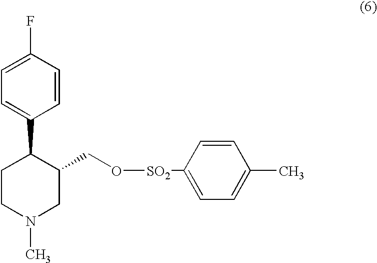
or a salt thereof.

2. The compound according to claim 1, wherein said compound is in a solid state.

3. The compound according to claim 2, wherein said compound is in a crystalline state.

4. The compound according to claim 3, wherein said crystalline compound contains 0.2% or less of a des-fluoro impurity thereof.

5. A process which comprises: reacting a compound of formula (1) or a salt thereof: 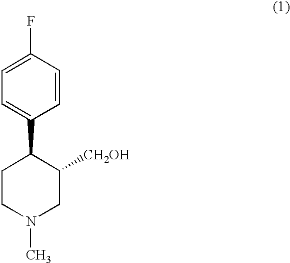
with a tosylate moiety-providing compound to form a compound of formula (6):

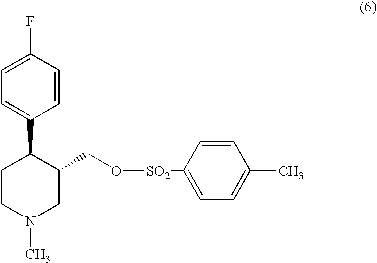

6. The process according to claim 5, wherein said tosyl moiety-providing compound is a tosyl-halide.

7. The process according to claim 6, wherein said tosyl-halide is p-toluene sulfonyl chloride.

8. The process according to claim 5, wherein said reacting is carried out in a solvent.

9. The process according to claim 8, wherein said solvent is ethyl acetate.

10. The process according to claim 8, wherein said reacting is carried out in the presence of an organic base.

11. The process according to claim 10, wherein said base is triethylamine or pyridine, and said base is present in an amount within the range of 1.0-1.5 times the molar amount of said tosyl moiety-providing compound.

12. The process according to claim 5, wherein said reacting is carried out at a temperature within the range of 0-80° C.

13. The process according to claim 5, which further comprises isolating said compound of formula (6) as a solid material.

14. The process according to claim 13, which further comprises crystallizing said solid compound of formula (6) from methanol, ethanol, or isopropanol.

15. The process according to claim 5, which further comprises converting said compound of formula (6) to paroxetine or a pharmaceutically acceptable salt thereof.

16. The process according to claim 15, wherein said converting comprises reacting said compound of formula (6) with sesamol to form methylparoxetine; reacting said methylparoxetine with phenylchloroformate to form a carbamate; and treating said carbamate with alkali to form paroxetine.

17. The process according to claim 16, which further comprises processing said paroxetine to form paroxetine hydrochloride or paroxetine mesylate.
